(19) United States
(12) Patent Application Publication
(10) Pub. No.: US 2003/0171232 A1
(43) Pub. Date: Sep. 11, 2003

(54) Decorative soap and method of making same

(76) Inventors: Scott H. Freeman, Huntington Beach, CA (US); Faith Freeman, Huntington Beach, CA (US)

Correspondence Address: STETINA BRUNDA GARRED & BRUCKER, 75 ENTERPRISE, SUITE 250, ALISO VIEJO, CA, 92656, US

(21) Appl. No.: 10/092,191

(22) Filed: Mar. 6, 2002

(51) Int. Cl. A61K 7/50

(52) U.S. Cl. 510/141; 510/142; 510/152; 510/147

(57) ABSTRACT

The present invention may be regarded as a decorative soap bar and a method for making same. The decorative soap bar includes a plurality of components that have a mineral-like coating. The components are encased in a non-opaque soap member.

### CROSS-REFERENCE TO RELATED APPLICATIONS

(Not Applicable)

### STATEMENT RE: FEDERALLY SPONSORED RESEARCH/DEVELOPMENT

(Not Applicable)

### BACKGROUND OF THE INVENTION

The present invention relates generally to soap, and more particularly to a decorative soap having a simulated raw stone aggregate appearance and a method for making same.

Decorative soaps are known in the art, and come in various shapes, sizes, colors and designs. For example, decorative soaps may include one or more colored shapes encased in a transparent or semi-transparent background soap material. The various shapes and colors can be chosen to match a consumer's particular tastes and/or decor. For example, the soap may include simple geometric shapes, such as squares rectangles or circles. Alternatively, the soap may include one or more complex shapes, for example, hearts, flowers, a cat, a Christmas tree, etc.

The decorative soaps described above may be formed by placing the decorative shape(s) in a soap bar mold and then pouring transparent or semi-transparent liquid soap over the decorative shape (s), allowing the soap to cool and solidify and removing the bar of soap from the mold. Alternatively, the soap can be formed into loaves using a similar process. The loaves can then be cut into bars of soap.

As mentioned above, soaps such as those described above may be matched to a consumer's decor. For example, a particular soap may be chosen because the decorative shapes are in colors that are complementary to a consumer's bathroom decor. However, even though such soaps may be complementary, they may not aesthetically blend as well as many consumers may like. For example, a consumer may have bathroom decor with a certain color tile. The consumer may desire soap that blends with the decor rather than detracts from it. For-example, a consumer may desire a soap that has a stone-like appearance that is similar to their bathroom floor or counter. Thus, a need exists for decorative soaps in various colors that have a natural appearance, such as a stone-like appearance.

### BRIEF SUMMARY OF THE INVENTION

An aspect of the present invention may be regarded as a decorative soap bar having a simulated raw stone aggregate appearance. The decorative soap bar includes a plurality of components that are covered with a mineral-like coating. The components are encased by a non-opaque soap background.

In accordance with other aspects of the invention, the decorative soap bar may be formed by cutting a decorative soap loaf such that surfaces of the components that are along a cut edge of the decorative soap bar do not possess mineral-like coating, and surfaces of the components that are not along the cut edge of the decorative soap bar, but rather remain encased by the non-opaque soap background, retain the mineral-like coating.

In accordance with yet other aspects of the invention, the mineral-like coating is a cosmetic grade, glitter-type metallic powder.

In accordance with still other aspects of the invention, the components are opaque. The components may be of multiple colors and/or multiple shapes.

In accordance with yet other aspects of the invention, the non-opaque soap member may be transparent. Alternatively, the non-opaque soap member may be semitransparent.

In accordance with still other aspects of the invention, the decorative soap bar may include a fragrance.

Another aspect of the present invention may be regarded as a method for making a decorative soap bar having a simulated raw stone aggregate appearance. A plurality of components are formed. The components are coated with a mineral-like coating. The coated components are placed in a mold. The mold is filled with a non-opaque liquid soap. The soap is solidified. The soap is removed from the mold.

In accordance with further aspects of the invention, the components are coated with the mineral-like coating by: (1) placing the components in a container; (2) placing the mineral-like coating substance in the container; (3) covering the container; (4) placing the covered container in a mixer; (5) tumbling the container in the mixer; and (6) removing the components from the container.

### BRIEF DESCRIPTION OF THE DRAWINGS

These as well as other features of the present invention will become more apparent upon reference to the drawings wherein:

FIG. 1 illustrates a prior art mixer;

FIG. 2 illustrates a mixer for tumbling components in order to coat the components in accordance with the present invention;

FIG. 3 illustrates a decorative soap bar having a simulated raw stone aggregate appearance formed in accordance with the present invention;

FIG. 4 is a flow diagram illustrating exemplary steps used to manufacture a decorative bar of soap, such as the one shown in FIG. 3;

FIG. 5 is a flow diagram illustrating exemplary steps used to coat components to be used to manufacture decorative bars of soap, such as the one shown in FIG. 3;

FIG. 6 illustrates uncoated components;

FIGS. 7-8 illustrate steps for coating the components of FIG. 6 in accordance with the present invention;

FIG. 9 illustrates coated components;

FIG. 10 illustrates creating a soap loaf including the coated components of FIG. 9; and

FIG. 11 illustrates slicing the loaf of FIG. 10 to create the decorative bar of soap shown in FIG. 3.

### DETAILED DESCRIPTION OF THE INVENTION

Aspects of the present invention include a decorative soap having a simulated raw stone aggregate appearance and a method of making same. Preferably, the soap is a transparent or semi-transparent bar of soap with opaque components encased within the bar of soap. The opaque components are coated with a powdered pigment. For example, the powdered pigment may be colored to resemble pyrite or chalcopyrite, which are also referred to as fool's gold. Preferably, the soap bar is formed by slicing a soap loaf. Thus, the components along the sliced edges will have a faceted, non-coated appearance which coupled with the coated components that are not along the cut edge creates the simulated raw stone aggregate appearance. As described in further detail below, a specialized mixer may be used to coat the components.

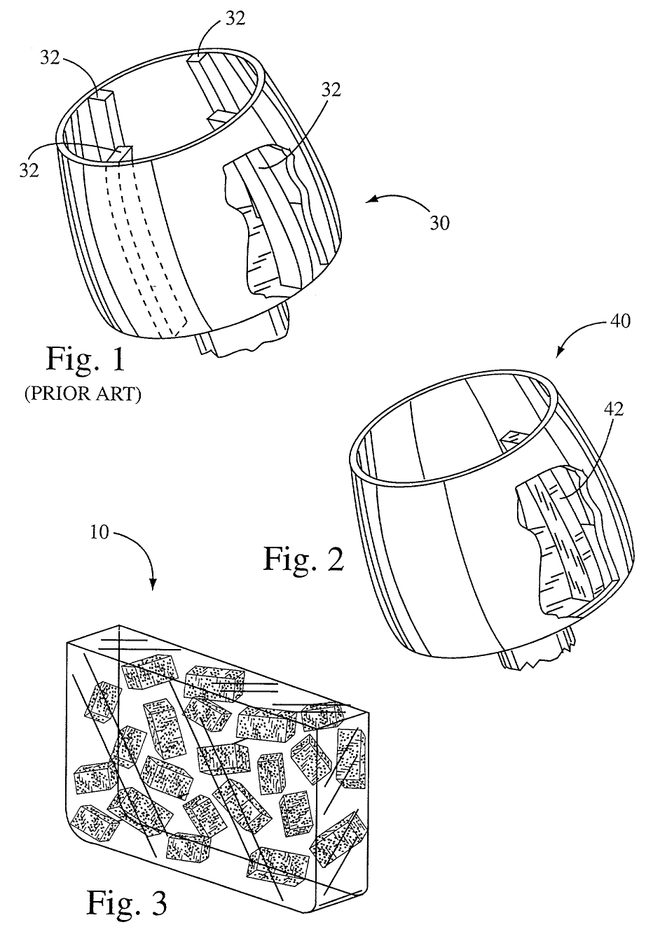

Referring now to the drawings wherein the showings are for purposes of illustrating preferred embodiments of the present invention only, and not for purposes of limiting the same, FIG. 1 illustrates a prior art mixer 30, such as a cement mixer. As can be seen, such mixers include a plurality of mixing blades 32.

FIG. 2 illustrates a mixer 40 used to coat components to create a decorative soap having a simulated raw stone aggregate appearance in accordance with the present invention. The mixer 40 shown in FIG. 2 is similar to the prior art mixer 30 shown in FIG. 1 except that it only has one mixing blade 42. Use of the mixer 40 shown in FIG. 2 is described in further detail later.

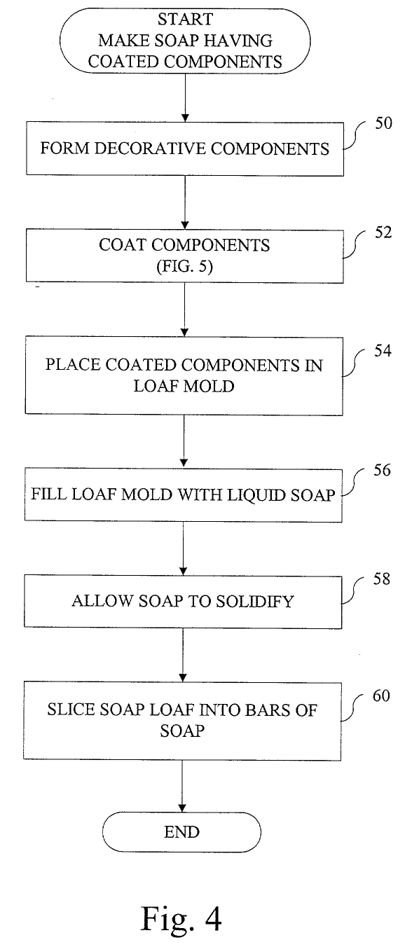

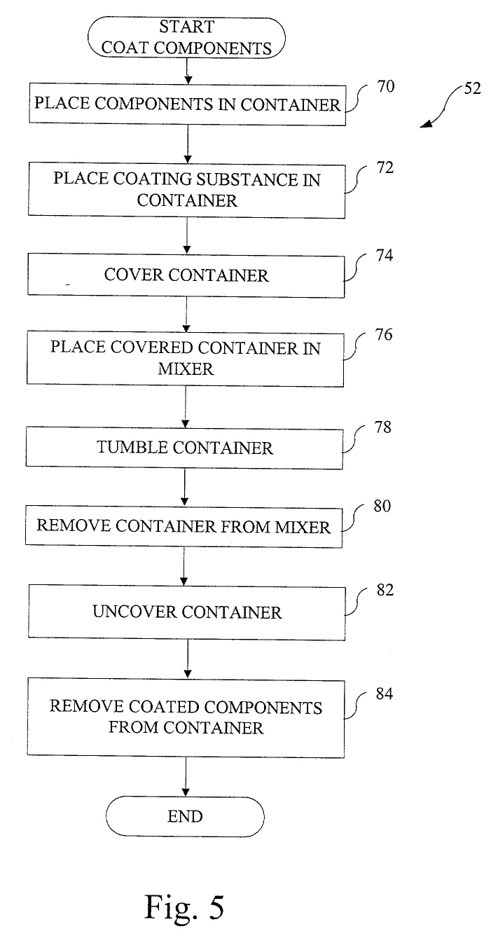

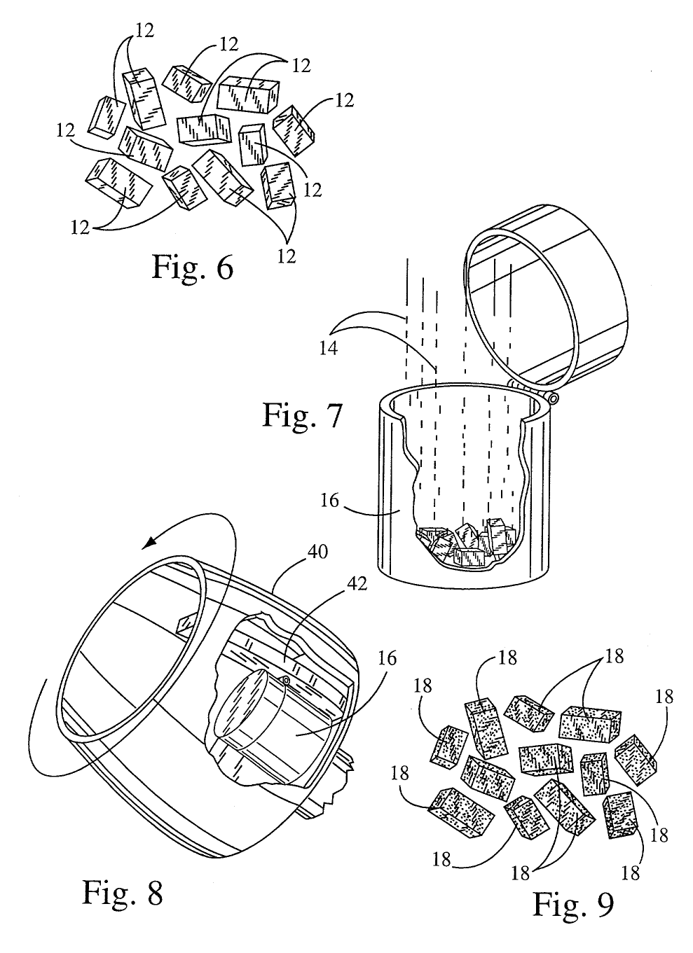

FIG. 3 illustrates a decorative soap 10 having a simulated raw stone aggregate appearance formed in accordance with the present invention. FIGS. 4-5 illustrate exemplary logic for manufacturing a decorative bar of soap, such as the one shown in FIG. 3. FIGS. 6-11 graphically illustrate various stages or steps of making the decorative bar of soap in accordance with the present invention. An exemplary method for manufacturing a decorative soap having a simulated raw stone aggregate appearance is described next with reference to the exemplary logic shown in FIGS. 4-5.

First, decorative components are formed (step 50 of FIG. 4). Exemplary components 12 are shown in FIG. 6. The soap components 12 may be formed using any known technique. For example, they may be made using molds or extruded through forming dies, or they may be made by creating a layer of soap and then die-cutting shapes into the layer of soap, etc. In exemplary embodiments, the components 12 are opaque structures preferably formed of glycerin soap material. The components 12 may be made of a single color or multiple colors. Likewise, the components can all be the same shape and size or they may be different shapes and sizes. Preferably, the components are various shapes and sizes and are in a variety of complementary colors so that they more accurately simulate stone structures occurring in nature.

Next, the components 12 are coated with a coating substance (step 52). Exemplary logic for coating the components in accordance with the present invention is shown in further detail in FIG. 5 and described below.

The coating process preferably comprise a dry process so that the soap components 12 do not dissolve. Preferably, a tumbling process as described in further detail below is used. Alternatives to a tumbling process include shaking the coating substance 14 onto the components 12 or dusting the coating substance 14 onto the components 12. However, shaking the coating substance 14 onto the components 12 is time consuming, does not create a uniform coating, and is dusty. If a dusting method is used, such as by using a flour sifter, the coating substance 14 likely will not sufficiently adhere to the components 12.

As shown in the tumbling process of FIG. 5, for personnel safety concerns, the shaped components are placed in a dust resistant container 16 (step 70) as shown in FIG. 7. For example, the container 16 may be a five-gallon plastic container. The coating substance 14 is placed in the container 16 (step 72). In exemplary embodiments, the coating substance is a cosmetic grade, glitter-type metallic powder.

As shown in FIG. 8, the container 16 is covered (step 74). In the illustrated embodiment, the container 16 includes a hinged lid. However, it will be appreciated that a container without a lid may be used as long as the container is covered. For example, a plastic bonnet resembling a shower cap may be used as a container cover. The container 16 is then placed in a mixer 40 (step 76), such as the one shown in FIG. 2. As described above with reference to FIG. 2, the mixer 40 used to tumble the components may be a prior art cement mixer 30 preferably modified such that all but one of the mixing blades 32 is removed. The mixer 40 having one blade 42 allows the container 16 to be raised and dropped within the mixer 40 with a sufficient amount of movement and force that allows the coating substance 14 to adhere to the components 12 without damaging either the components 12 or the container 16. Preferably, the tumbling process is performed at an ambient temperature. The container 16 holding the components 12 and the coating substance 14 should be tumbled for a sufficient amount of time to coat the components 12 (step 78). In exemplary embodiments, the tumbling time is between about 10 seconds and 60 seconds, preferably about 20 seconds. The amount of tumbling time required to coat the components 12 depends on the tumbling speed, as well as the quantity of components 12 in the container 16. It will be appreciated that in addition to coating the components 12, the tumbling process causes the surfaces of the components to have more of a stone-like appearance due to the movement of the components during the tumbling process.

Once the components 12 are coated, the container 16 is removed from the mixer 40 (step 80). The container 16 is then uncovered (step 82) and the coated components 18 are removed from the container 16 (step 84). FIG. 9 illustrates the components shown in FIG. 6 after they have been coated.

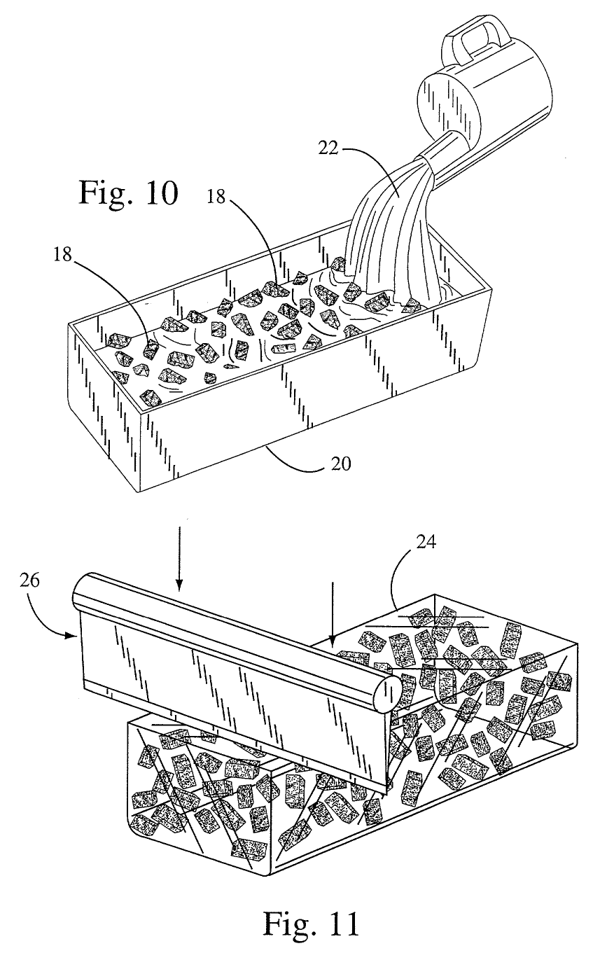

After the components 12 are coated, the coated components 18 are placed in a mold 20 (step 54 of FIG. 4). After the coated components 18 are placed in the mold 20, liquid soap 22 preferably transparent and/or semitransparent glycerin soap is poured into the mold 20 (step 56) until the mold 20 is filled, as shown in FIG. 10. It will be appreciated that the liquid soap 22 may have a fragrance. The coated components 18 may have a fragrance in addition to or instead of the liquid soap 22 having a fragrance, if desired. The liquid soap 22 is then allowed to solidify (step 58). For example, the liquid soap may be molten and may solidify when cooled.

After the soap 22 has solidified in the mold 20, the soap is removed from the mold 20. If the mold is a soap loaf mold, the soap loaf 24 may be sliced to create bars of soap (step 60). In exemplary embodiments, the mold 20 is a loaf mold. For example, the loaf mold may be twelve inches in length, twelve inches in width, and three inches in height. In exemplary embodiments, the soap loaf 24 is cut to create multiple smaller loaves. For example, a soap loaf that is twelve inches in length, twelve inches in width, and three inches in height may be cut to create two loaves that are each twelve inches in length, six inches in width, and three inches in height. As shown in FIG. 11, the soap loaves 24 may be cut with a cutting device knife or guillotine 26 to form bars of soap, such as the soap bar 10 shown in FIG. 3.

When cut, the cut edges of the coated components 18 of the soap bar 10 have a faceted, non-coated appearance which simulates a facet cut formed on natural stone. Thus, the present invention can be used to create decorative soap bars 10 that have a stone-like or gem-like appearance with the encased outer edges being coated and the cut edges not having a coating and having a faceted appearance.

While an illustrative and presently preferred embodiment of the invention has been described in detail herein, it is to be understood that the inventive concepts may be otherwise variously embodied and employed and that the appended claims are intended to be construed to include such variations except insofar as limited by the prior art.

What is claimed is:

1. A decorative soap bar having a plurality of outer surfaces, the decorative soap bar comprising:
a plurality of components, each component having a plurality of surfaces covered with a mineral-like coating; and
a non-opaque soap background substantially encasing the components.

2. The decorative soap bar of claim 1, wherein the decorative soap bar is formed by cutting a decorative soap loaf such that surfaces of the components that are coplanar with a respective outer surface of the decorative soap bar do not have the mineral-like coating, and surfaces of the opaque components that are encased by the non-opaque soap background retain the mineral like coating.

3. The decorative soap bar of claim 1, wherein the mineral-like coating is a cosmetic grade, glitter-type metallic powder.

4. The decorative soap bar of claim 1, wherein the components are opaque.

5. The decorative soap bar of claim 1, wherein the plurality of components are a plurality of colors.

6. The decorative soap bar of claim 1, wherein the plurality of components are a plurality of shapes.

7. The decorative soap bar of claim 1, wherein the non-opaque soap member is transparent.

8. The decorative soap bar of claim 1, wherein the non-opaque soap member is semi-transparent.

9. The decorative soap bar of claim 1, further comprising a fragrance.

10. A method of forming a decorative soap bar comprising:
a) forming a plurality of components, each of the components having a plurality of surfaces;
b) coating the surfaces of the components with a mineral-like coating substance;
c) placing the coated components in a mold;
d) filling the mold with a non-opaque liquid soap;
e) solidifying the non-opaque liquid soap; and
f) removing the solidified soap from the mold.

11. The method of claim 10, (b) comprises:
i) placing the components in a container;
ii) placing the mineral-like coating substance in the container;
iii) covering the container;
iv) placing the covered container in a mixer;
v) tumbling the container in the mixer; and
vii) removing the components from the container.

12. The method of claim 10, wherein the mold is a loaf mold, and further comprising:
g) slicing the loaf to form a decorative soap bar.

13. The method of claim 10, wherein the mineral-like coating is a cosmetic grade, glitter-type metallic powder.

14. The method of claim 10, wherein the components are opaque.

15. The method of claim 10, wherein the plurality of components are a plurality of colors.

16. The method of claim 10, wherein the plurality of components are a plurality of shapes.

17. The method of claim 10, wherein the non-opaque soap member is transparent.

18. The method of claim 10, wherein the non-opaque soap member is semi-transparent.

19. The method of claim 10, further comprising a fragrance.
